(19) United States
(12) Patent Application Publication
(10) Pub. No.: US 2024/0009125 A1
(43) Pub. Date: Jan. 11, 2024

(54) NITRIC OXIDE FORMULATION DELIVERY DEVICE AND METHOD OF USE

(71) Applicant: NOxy Health Products, Inc., San Mateo, CA (US)

(72) Inventors: C. Michael Miller, Anaconda, MT (US); David A, Bell, Farmington, UT (US)

(21) Appl. No.: 18/349,026

(22) Filed: Jul. 7, 2023

Related U.S. Application Data
(60) Provisional application No. 63/368,149, filed on Jul. 11, 2022.
(60) Provisional application No. 63/396,859, filed on Aug. 10, 2022.

(51) Int. Cl. A61K 9/12; A61K 33/00

(52) U.S. Cl. CPC A61K 9/122; A61K 33/00; A61K 9/0014

(57) ABSTRACT

A system for producing a gas-filled medium comprises a first pressurized vessel containing a gas, a second vessel containing a surfactant solution, a dose chamber in fluid communication with the first pressurized vessel, a mixing chamber in fluid communication with the dose chamber and the second vessel, the mixing chamber being configured to mix the gas flowing out of the dose chamber and the surfactant solution flowing out of the second vessel to produce a gas-filled solution, and a foaming nozzle comprising mesh screens configured to convert the gas-filled solution into a gas-filled foam medium, the foaming nozzle configured to discharge the gas-filled foam medium.

### CROSS-REFERENCE TO RELATED APPLICATIONS

This U.S. patent application claims priority under 35 U.S.C. § 119(e) to U.S. Provisional Application 63/368,149, filed on Jul. 11, 2022 and U.S. Provisional Application 63/396,859, filed on Aug. 10, 2022. The disclosures of these prior applications are hereby incorporated by reference in their entireties.

### FIELD

The present disclosure relates generally to a system for incorporation of gases into various mediums, and to the production of a medium that contains gases that may be used for various purposes. For example, the present disclosure relates to a system for producing nitric oxide-containing mediums for treating affected areas of skin and wounds, wherein the gas-containing mediums may be used to treat a patient through a topical skin therapy, such as, for example, to promote healing of chronic wounds, or to provide exogenous nitric oxide supplementation.

### BACKGROUND

This section provides background information related to the present disclosure which is not necessarily prior art.

Nitric Oxide (NO) is a molecular compound, that is naturally occurring throughout the body, and when available in sufficient concentration plays a key role in the dynamic “Molecular and Cellular Skin Wound Healing Processes.”

In its initial formulation in the epithelial layers of all blood vessels, this endogenous enzyme, or Endothelial Nitric Oxide Synthase, “eNOS,” formulates as a “signaling molecule” where it is dispensed by vascular flows and can motivate important cellular functions found in the “Vascular Response Cascade” associated with wound healing, including vasodilation, contraction and clotting, closure, and nitric oxide enzyme-guided, protective inflammation. When appropriately activated and sustained by a sufficient concentration of enzymatic nitric oxide, it is a powerful promotor of improved oxygenation and nutrient delivery, as well as an anti-pathogenic within the biosphere of wounded tissues.

Likewise, when stimulated by excessive inflammation, NO is actively unregulated by “iNOS,” where it can terminate inflammatory neutrophilic and macrophagic functions and actively target and dismantle pathogens associated with chronic wound development leading up to scar formation, renewed cellular growth, and epithelial healing requisite to wound contraction and wound scar remodeling.

But sometimes due to age, and/or concomitant disease, the body lacks sufficient NO concentration to produce and deliver sufficient synthase-driven nitric oxide on its own in order to overcome the initial stages of increasing inflammation which lead to chronic wounding. In such cases, an alternative exogenous NO supplementation may be required.

Even so, NO supplementation poses significant challenges primarily associated with its short half-life (often reported as less than 3 seconds), and its proclivity to instantaneously react with ambient oxygen to form Nitrogen Dioxide (NO₂), which in turn can further complicate the storage, transportation, and wound site application of NO. Moreover, methods for motivating the diffusion of NO directly into the wound poses significant challenges.

### SUMMARY

This section provides a general summary of the means and methods associated with the invention and is not a comprehensive disclosure of its full scope or of all its features.

In one implementation, a method for producing a gas-filled medium comprises providing a source of gas, providing a surfactant solution, mixing the gas and the surfactant solution in a manner that produces a gas-filled medium, and applying the gas-filled medium topically to the skin.

In some implementations, the medium may be a cluster of bubbles. The bubbles may comprise at least one of macro-bubbles, micro-bubbles, nano-bubbles or pico-bubbles. The source of the gas may be a pressurized vessel. The gas may comprise one or more of nitric oxide, oxygen, or nitrogen. The surfactant may be selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.

In another implementation, a method for producing a gas-filled medium comprises providing a source of gas, providing a first solution containing at least Acidic Liquid, Surfactant, and Purified Water, providing a second solution containing at least a Surfactant, Nitrite, and Purified Water, using the pressure difference from source of gas to draw out a specific volume of the first and second solutions and enter a holding chamber, allowing the first and second solutions to mix in the holding chamber and trap the gas, opening a valve to release the mixed first and second solutions and trapped gas, and applying the gas-filled medium topically to the skin.

In some implementations, the medium may be a cluster of bubbles. The bubbles may comprise at least one of macro-bubbles, micro-bubbles, nano-bubbles or pico-bubbles. The source of the gas may be a pressurized vessel. The gas may comprise one or more of nitric oxide, oxygen, or nitrogen. The surfactant may be selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.

In another implementation, a system for producing a gas-filled medium comprises a first pressurized vessel containing a gas, a second vessel containing a surfactant solution, a dose chamber in fluid communication with the first pressurized vessel, a mixing chamber in fluid communication with the dose chamber and the second vessel, the mixing chamber being configured to mix the gas flowing out of the dose chamber and the surfactant solution flowing out of the second vessel to produce a gas-filled solution, and a foaming nozzle comprising mesh screens configured to convert the gas-filled solution into a gas-filled foam medium, the foaming nozzle configured to discharge the gas-filled foam medium.

In some implementations, the medium may be a cluster of bubbles. The bubbles may comprise at least one of macro-bubbles, micro-bubbles, nano-bubbles or pico-bubbles. The source of the gas may be a pressurized vessel. The gas may comprise one or more of nitric oxide, oxygen, or nitrogen. The surfactant may be selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.

The mixing chamber may comprise an eductor configured to receive the gas flowing out of the dose chamber and the surfactant solution flowing out of the second vessel.

The system may further comprise a first valve disposed between the first pressurized vessel and the dose chamber, a second valve disposed between the dose chamber and the mixing chamber, and a third valve disposed between the second vessel and the mixing chamber.

The second vessel may be pressurized.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected configurations and not all possible implementations and are not intended to limit the scope of the present disclosure.

FIG. 1 is a schematic of an exemplary system for nitric oxide administration;

FIG. 2 is a schematic of an exemplary system for nitric oxide administration;

FIG. 3 is a schematic of an exemplary system for nitric oxide administration;

FIG. 4 is a schematic of an exemplary system for nitric oxide administration;

FIG. 5 is a schematic of an exemplary system for nitric oxide administration;

FIG. 6 is a schematic of an exemplary system for nitric oxide administration;

FIG. 7 is a schematic of an exemplary system for nitric oxide administration; and

FIG. 8 is a schematic of an exemplary system for nitric oxide administration.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

According to some embodiments, the present disclosure comprises a system for using any suitable combination of one or more surfactant solutions, or other suitable mediums, and nitric oxide, to produce a desired nitric oxide-containing medium, alternatively referred to herein as an NO-containing medium. For example, and not by way of limitation, according to some embodiments, the source of nitric oxide may be a pressurized gas cylinder or tank, or the like, capable of maintaining a pressure of at least about 1 psig to about 900 psig. According to some embodiments, a surfactant solution comprises one or more surfactants that are adapted and/or configured to produce a desired medium, or foam, and is compatible with the nitric oxide.

In some embodiments, surfactants that may be used include neutral, anionic, or cationic types of surfactants, referring to the electrical charge the surfactant molecules tend to acquire when dissolved in liquid. Examples of neutral surfactants are cocamide monoethanolamine (Cocamide MEA), cocamide diethanolamine (Cocamide DEA), fatty alcohol ethoxylates, amine oxides, and sulfoxides. Examples of anionic or negatively charged surfactants are sodium lauryl sulfate (SLS), sodium laureth sulfate (SLES), ammonium lauryl sulfate (ALS), ammonium laureth sulfate (ALES), sodium stearate, and potassium cocoate.

In some embodiments, the cationic surfactants is/are selected from coco betaine, cocamidopropyl betaine (CAPB), and cetyltrimethylammonium bromide. Generally, the media or solvent may be water, preferably purified, such as distilled or deionized water, to provide a consistent final surface tension for the surfactant.

According to some embodiments, the nitric oxide-containing medium may comprise nitric oxide bubbles and perhaps a biologically inert gas such as nitrogen or carbon dioxide to adjust the NO concentration in the gas phase. According to some embodiments nitric oxide bubbles may be micro-sized bubbles, nano-sized bubbles, and/or pico-sized bubbles. The NO-containing medium may also comprise one or more additional media that are suitable for the intended purpose of treating wounds, for instance, including without limitation, solutions, gels, foams, thixotropic fluids, and the like, such as, for example, med-honey, benzalkonium chloride, shea butter, fragrance, hyaluronic acid, and the like.

According to some embodiments, the nitric oxide-containing media may be a foam that comprises layers of bubbles, wherein the bubbles are selected from the group consisting of micro-sized bubbles, nano-sized bubbles, pico-sized bubbles, or mixture or combinations thereof.

In an embodiment, the bubbles are macro-sized bubbles. In another embodiment, the bubbles are pico-sized bubbles. In a further embodiment, the bubbles are a mixture of micro-sized bubbles, nano-sized bubbles, and pico-sized bubbles. In a further embodiment, the mixture comprises approximately a distribution of bubbles based on size of micro to pico meter diameter.

In a further embodiment, increasing the amount of a suitable surfactant (to a point) in a surfactant solution, or any suitable medium, may allow for production of smaller nitric oxide bubbles, micro-sized bubbles, nano-sized bubbles, and/or pico-sized bubbles.

In some embodiments, the nitric oxide-containing media may resemble shaving cream.

In another embodiment, an alternative source of nitric oxide gas may be employed. The source of nitric oxide may be, as an alternative to a pressurized gas cylinder or tank, from a chemical reaction that produces nitric oxide. Such reactions may include acidified nitrite, ammonia oxidation, plasma oxidation of nitrogen gas, etc. According to some embodiments, sources of nitric oxide that contain a certain percentage of nitrogen, or other similar gases, may also be used. According to some embodiments, the source of gas may provide a driving element for the foam-producing system, a medicinal element, or both.

In some embodiments, the system for providing a gas-filled foam comprises a pressurized gas cylinder and one or more surfactants, and/or one or more sources of surfactants. The gas cylinder may be any suitable container that can provide the desired gas. The gas may be any suitable gas that can be used to form bubbles. For example, and not by way of limitation, the gas may be one or more of nitrogen, argon, oxygen, nitric oxide, nitrous oxide, or the like.

In some embodiments, the one or more sources of surfactants may include any suitable container, pressurized or non-pressurized, and may be any suitable surfactant.

In additional embodiments, the suitable surfactants may also comprise other components, such as glycerin, and/or corn syrup to control and/or manage the size and formulation and structure of bubbles. Other components that optionally may be used may be selected form the group consisting of Ceramides, Essential fatty acids, glycerin, glycols, polyols, trimethylol propane, triethanolamine, pentaerythritol, sorbitol, or sucrose, polyethylene glycol (PEG),polyethylene oxide (PEO), polyoxyethylene (POE) hyaluronic acid, sodium PCA, vitamin C, vitamin E, vitamin A, coenzyme Q10, and fragrances such as lemon, lavender, tea tree oil, lemon grass, vanilla, rose, peppermint, and the like. The sizes of pressurized gas cylinders and sources of surfactant may be varied to accommodate different circumstances, such as a need for portability or for the ability to produce a greater volume of a gas-filled foam.

In some embodiments, pressurized nitric oxide may flow from a first pressure vessel through a metering needle valve and into a mixing device. Any number of mixing devices may be used, including an eductor. In one embodiment, the mixing device is an eductor.

In some embodiments, pressurized nitric oxide may flow through a surfactant (e.g., in a container or any other suitable vessel) to create a nitric oxide-containing foam.

In a further embodiment, the pressurized nitric oxide is mixed in the mixing device such as an eductor with a pressurized stream of water, a surfactant (such as coco betaine), and a carrier gas (such as nitrogen) from a second pressure vessel. The mixture of the pressurized nitric oxide, the pressurized stream of water, the surfactant (such as coco betaine), and the carrier gas then pass through a mixing and directing nozzle. The resultant mixture is emitted to the ambient air as foam comprising micro-bubbles that may be topically applied to an open skin wound or burn. The resultant mixture exhibits medicinal properties and acts as a localized antimicrobial agent, vasodilator, and analgesic.

According to a further embodiment, a pressurized gas cylinder is fluidly connected and/or coupled to a chamber. Any suitable connection between the gas cylinder and the chamber may be used. Likewise, any suitable connection between the chamber and the eductor may be used. According to some embodiments, any suitable connection between the surfactant source and the eductor may be used. According to some embodiments, suitable connection devices include “quick-connect” type connections, threaded connections, etc., between hoses and/or tubes. According to some embodiments, suitable connections may include a pressure regulator and/or equipment providing a choked flow. According to some embodiments, one or more pressurized gas cylinders and/or surfactant tanks may be suitably connected to existing gas sources, depending on the intended use and available equipment at a given site.

According to some embodiments, upon a first activation action, the gas from the pressurized cylinder may fill the chamber. Upon a second activation action, the gas from the chamber is allowed to pass through an eductor, which passage through the eductor draws the surfactant from its source sending the gas-surfactant mixture through a nozzle producing a gas-filled foam. According to some embodiments, systems, methods, and applications, the nozzle may include a screen or other configuration to promote the formation of the foam.

According to some embodiments, systems, methods, and applications, a resultant foam generally comprises a plurality of bubbles. In some embodiments, bubble wall thickness may be controlled and/or manipulated for a desired application. According to some embodiments, systems, methods, and applications, bubbles from pico-sized or larger may be useful for different situations and intended purposes. In some embodiments, systems, methods, and applications, bubbles may be configured to facilitate collapsing. In some embodiments, systems, methods, and applications, bubbles may be configured to resist collapsing. According to different implementations, bubbles may or may not be collapsible depending on physical factors, different situations, and/or intended purposes.

According to some embodiments, systems, methods, and applications, a gas may be configured and adapted to be delivered for topical use. According to some embodiments, systems, methods, and applications, a gas may be configured and adapted to be delivered for transdermal absorption of the gas. For example, and not by way of limitation, according to some embodiments, systems, methods, and applications, an argon gas-filled foam may be applied to a burn wound on a person's skin in such a manner that oxygen is excluded from the surface of the wound. The lack of oxygen may keep pain receptors from firing, thus helping to alleviate pain of the wound. According to some embodiments, systems, methods, and applications, a nitrous oxide gas-filled foam may be applied as a topical analgesic. In other circumstances, according to some embodiments, systems, methods, and applications, an oxygen gas-filled foam may be applied to a wound to provide an oxygen enrichment process for the wound. According to some embodiments, systems, methods, and applications, a nitric oxide gas-filled foam may be applied to a wound to promote a healing response.

According to some embodiments, systems, methods, and applications, a gas may be produced in a variety of ways, for example, through one or more chemical reactions, etc. No matter the source of the gas, or the type of the gas, according to some embodiments, systems, methods, and applications, a gas-filled foam may be produced and used as a delivery mechanism for the gas. According to some applications, the delivery mode is topical, but other delivery types are also possible. According to some applications, a bubble is considered a sterile, transport mechanism for the gas inside the bubble.

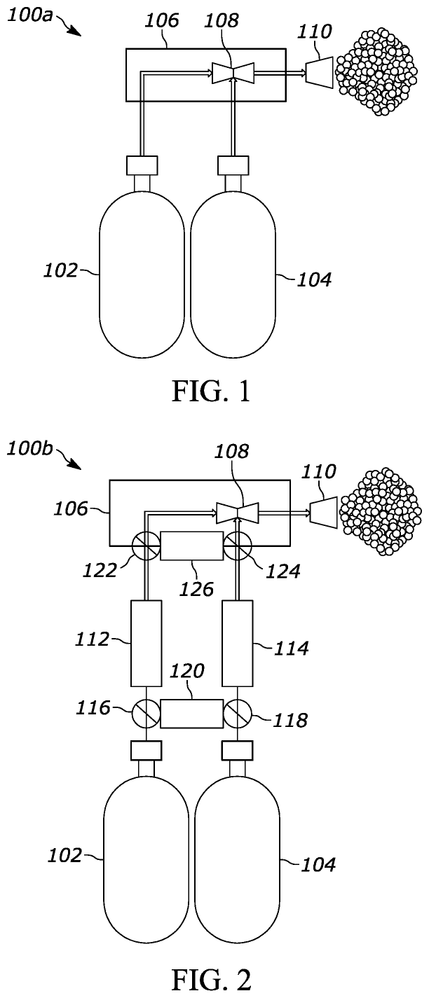

Referring to FIGS. 1-8, several exemplary embodiments of a system for nitric oxide administration 100a-h are generally shown. The systems 100a-h described herein may include common features that are represented by common reference numerals. It should be understood that various systems 100a-h may be suitably combined with one another as understood to a skilled artisan. For example, one or more features of an exemplary system may be incorporated into another exemplary system as suitable. As another example, one or more features of an exemplary system may be omitted based on the teachings of another exemplary system.

Referring to FIG. 1, a first exemplary system for nitric oxide administration 100a is generally shown. The system 100a comprises a first container 102 and a second container 104. In some implementations, the containers (or vessels) 102, 104 are pressurized. In other implementations, the first container 102 is pressurized and the second container 104 is not pressurized. The first container 102 may contain a first mixture comprising pure nitric oxide or nitric oxide mixed with an additional inert gas propellant such as nitrogen gas. The second container 104 may contain a second mixture comprising a mixture of surfactant and other materials for producing a foam when mixed with the first containment gas(es) including water and a propellant. The system 100a includes a mixing region or chamber 106 in fluid communication with the containers 102, 104. The mixing region 106 may include an eductor 108 configured to receive the first mixture and the second mixture. The eductor 108 is configured to create a suction force to receive the second mixture from the second container 104. In some implementations, the eductor 108 is a Venturi eductor. Both the first and second container flows are initiated, controlled, combined, and mixed in the mixing region 106, and then released through a foaming nozzle 110 of the system 100a to produce a rich foam not unlike shaving cream that is made up of microbubbles containing a majority of pure nitric oxide. The foam, also referred to as a gas-containing medium, may then be topically applied to a wound.

Referring to FIG. 2, a second exemplary system for nitric oxide administration 100b is generally shown. The second system 100b is generally the same as the first system 100a except as described below.

The second system 100b includes a first precision dose chamber 112 in fluid communication with the first container 102 and a second precision dose chamber 114 in fluid communication with the second container 104. The second system 100b includes a first nominally-closed (NC) influent valve 116 disposed between the first container 102 and the first precision dose chamber 112 and a second NC influent valve 118 disposed between the second container 104 and the second precision dose chamber 114. The first NC influent valve 116 is connected to the second NC influent valve 118 by a first valve control 120 that controls the valves 116, 118 to open and close both valves 116, 118 simultaneously.

The second system 100b includes a first NC effluent valve 122 disposed between the first precision dose chamber 112 and the mixing region 106 and a second NC effluent valve 124 disposed between the second precision dose chamber 114 and the mixing region 106. The first NC effluent valve 122 is connected to the second NC effluent valve 124 by a second valve control 126 that controls the valves 122, 124 to open and close both valves 122, 124 simultaneously.

Effluent lines from both the first and second containers 102, 104 provide respective flows through the first and second NC influent valves 116, 118 into the precision dosage chambers 112, 114 that are in turn secured against loss by the first and second NC effluent valves 122, 124. The first and second NC influent valves 116, 118 are simultaneously opened by a priming mechanism (not shown), permitting flow into each of the precision dose chambers 112, 114 until the precision dose chambers 112, 114 are completely filled at their respective containment's pressures and temperatures. Once the precision dose chambers 112, 114 are filled, the first and second NC influent valves 116, 118 are closed, trapping their respective containment's contents.

Nitric oxide microbubble foam production is initiated when the first and second NC effluent valves 122, 124 are simultaneously opened permitting a combining and mixing of the effluent flows at the mixing region 106 prior to the flow passing through the foaming nozzle 110 into the ambient.

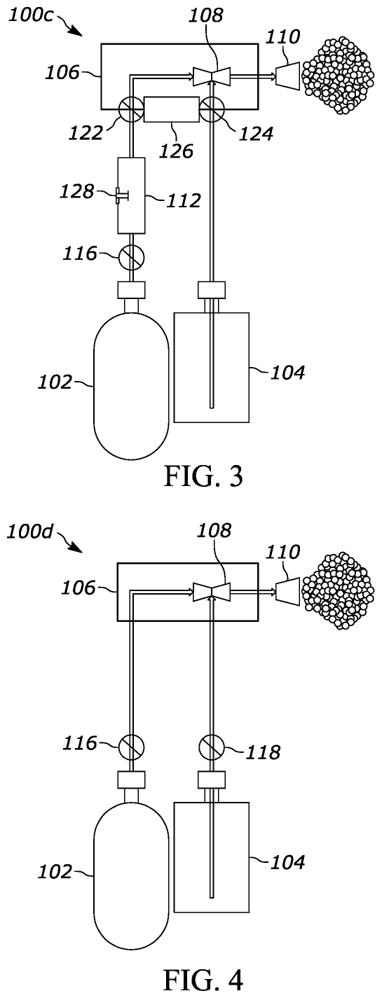

Referring to FIG. 3, a third exemplary system for nitric oxide administration 100b is generally shown. The third system 100c is generally the same as the second system 100b except as described below.

Compared to the second system 100b, the third system 100c may omit the second precision dose chamber 114, the second NC influent valve 118, and the first valve control 120. The third system 100c may include a mechanical fill indicator 128 in the first precision dose chamber 112 that indicates a full charge of the first precision dose chamber 112. The second container 104 may be unpressurized and the second mixture may be a liquid mixture.

When the first NC influent valve 116 is opened, an effluent line from the first container 102 provides pressurized flow into the first precision dose chamber 112. The first NC effluent valve 122 prohibits flow of NO gas from the first precision dose chamber 112 during the filling process. The first precision dose chamber 112 is filled, and the first influent NC valve 116 is allowed to close, securing the “charge” of NO gas in the first precision dose chamber 112. The mechanical fill indicator 128 may be forced outward from the wall of the first precision dose chamber 112 indicating a “full charge.”

The first and second NC effluent valves 122, 124 may be simultaneously opened permitting gas flow from the first precision dose chamber 112 into the motive opening of the eductor 108. This motive gas flow induces a suction flow in the effluent line of the second container 104, up through the open second NC effluent valve 124 and into the suction opening of the eductor 108 where the first mixture is mixed with the second mixture through the eductor 108. Nitric oxide microbubble foam production may be initiated when motive gas and the suction liquid flows enter and pass through the eductor 108. A further combining and mixing of the flows may occur as the mixed flow passes through the foaming nozzle 110 and into the ambient.

Referring to FIG. 4, a fourth exemplary system for nitric oxide administration 100d is generally shown. The fourth system 100d is generally the same as the first system 100a except as described below.

Compared to the first system 100a, the fourth system 100d may include the first and second NC influent valves 116, 118. The second container 104 may be unpressurized and the second mixture may be a liquid mixture.

When the second NC influent valve 118 is open and the first NC influent valve 116 is opened, nitric oxide gas flow through the gas effluent line from the first container 102 as a motive gas flow through the eductor 108. The motive gas flow induces a suction flow in the effluent line of the unpressurized liquid second container 104, up through the open second NC influent valve 118 and into the suction opening of the eductor 108 where the liquid is mixed with the gas flow passing through the eductor 108. Nitric oxide microbubble foam production may be initiated when motive gas and the suction liquid flows enter and pass through the eductor 108. A further combining and mixing of the flows may occur as the mixed flow passes through the foaming nozzle 110 and into the ambient.

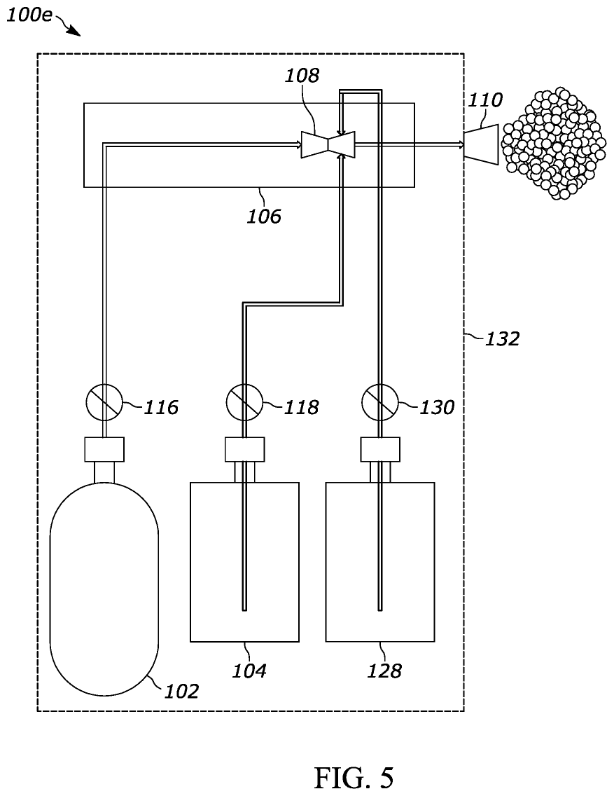

Referring to FIG. 5, a fifth exemplary system for nitric oxide administration 100e is generally shown. The fifth system 100e is generally the same as the fourth system 100d except as described below.

Compared to the fourth system 100d, the fifth system 100e may include a third container 128 and a third NC influent valve 130 between the third container 128 and the mixing region 106. The fourth system 100d may be integrated into a single handheld device 132 taking any suitable form and shape.

The first container 102 may be a pressurized container that holds a propellant gas such as CO2 and may be a standardized cartridge. The second and third containers 104, 128 may be liquid containers that may or may not be pressurized. The second and third containers 104, 128 may not be pressurized and may hold Formula A and Formula B liquid mixtures, respectively, for producing a rich, NO microbubble foam when mixed with the gases of the first container 102 in the suction portion of the eductor 108.

According to the operation of the fifth system 100e, the second and third NC influent valves 118, 130 are opened. Then, the first NC influent valve 116 may be opened, initiating motive gas flow through the gas effluent line from the first container 102 as a “motive gas flow” into and through the eductor 108. Motive gas flow induces a suction flow in the effluent lines of the second and third containers 104, 128, drawing Formula A and Formula B liquids up through the open suction valves and into the suction openings of the eductor 108 where the liquid is turbulently mixed with the gas flow passing through the eductor 108. The Venturi section of the eductor 108 creates a low-pressure suction area in the nozzle of the eductor 108. Nitric oxide microbubble foam production is initiated when motive gas and the suction liquid flows enter, mix, and pass through the eductor 108 into the foaming nozzle 110.

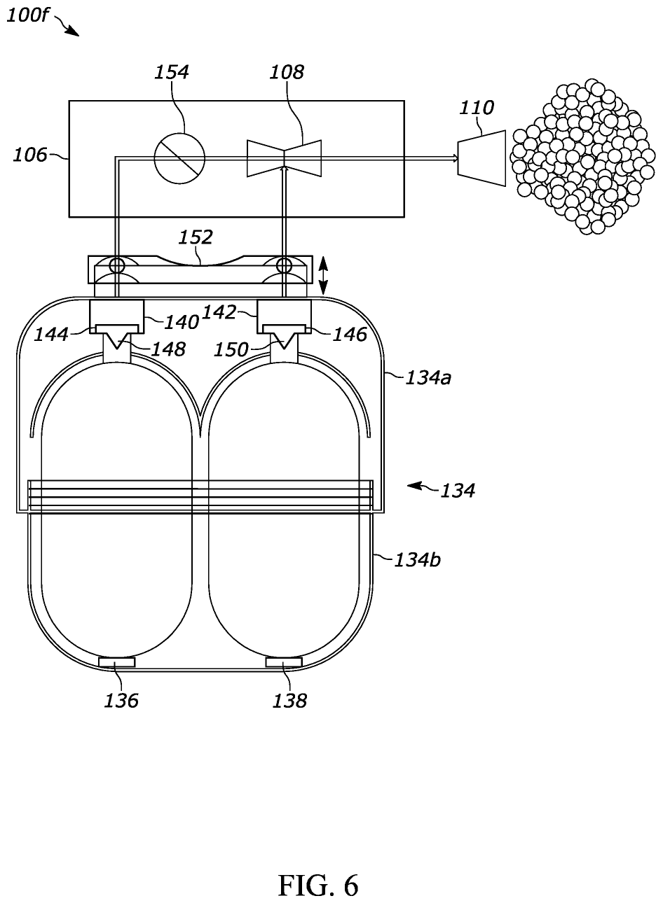

Referring to FIG. 6, a sixth exemplary system for nitric oxide administration 100f is generally shown. The system 100f may include a vessel housing 134 comprising a top portion 134a and a bottom portion 134b configured to receive the first and second containers 102, 104.

The first pressurized container 102 contains the nitric acid source and an inert gas such as nitrogen and the second container 104 contains a surfactant solution in water and an inert gas such as nitrogen. The top and bottom portions 134a, 134b may be sealed together using tightening threads, which allow for re-use of the vessel housing 134. That is, when the contents of the first and second pressurized containers 102, 104 are consumed, the vessel housing 134 may be dissembled by loosening the tightening screws, removing the empty pressurized vessels, replacing the consumed pressurized vessels with full pressurized vessels, and reassembling the vessel housing 134 by screwing the top and bottom pieces 134a, 134b together.

The interior of the vessel housing 134 may include two rubber bumpers 136, 138 on the bottom part for holding the first and second containers 102, 104 in place. The vessel housing 134 may include first and second caps 140, 142, each with a seating gasket 144, 146 and a puncture needle 148, 150 that fit snugly on top of the first and second containers 102, 104.

The vessel housing 134 is further equipped with a foam ejector and push valve assembly 152 that is connected to the exterior of the top part 134a. Attached to the foam ejector and push valve assembly 152 is a needle valve assembly 154 that is capable of limiting the flow of gas to a precise volume. The needle valve assembly 154 may also include the eductor 108 and the foaming nozzle 110.

In operation, each pressurized container 102, 104 is inserted into its respective molded holder of the vessel housing 134 and the top portion 134a is secured to the second portion 134b via tightening forces, which force each container 102, 104 into rupture contact with the puncture needle valves 148, 150 and opens and seals the containers 102, 104 against the seating gaskets 144, 146. The foam ejector and push valve assembly 152 opens both container flow pathways into the mixing region 106 which both mixes the flows from both vessels made up of a precise limited amount of nitric oxide and surfactant-laden water sufficient to create a rich shaving cream like foam when passed through the foaming nozzle 110. A mix of nitric oxide and nitrogen is contained within each micro-bubble of the foam and each bubble is comprised of water and the inert surfactant. The foam may then be placed over the wound area.

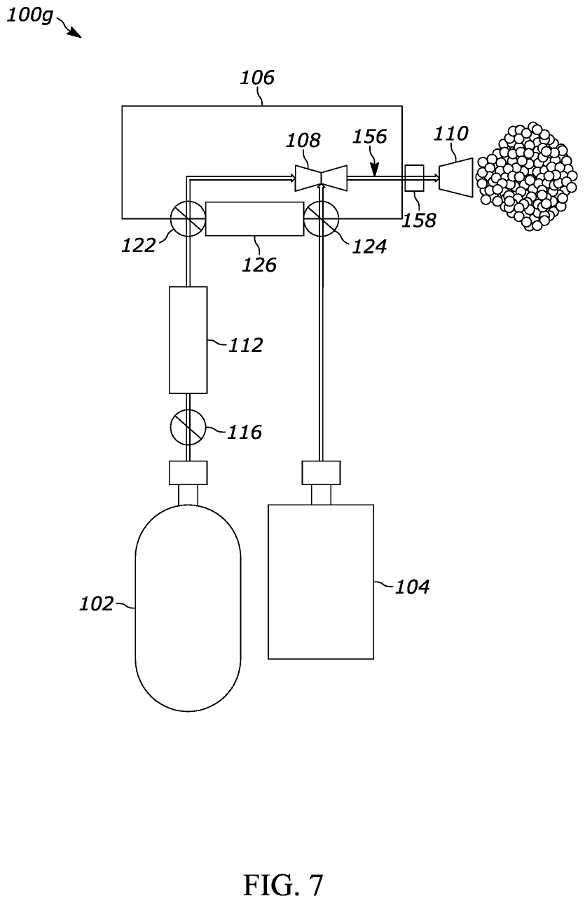

Referring to FIG. 7, a seventh exemplary system for nitric oxide administration 100g is generally shown. The seventh system 100g is generally the same as the third system 100c except as described below.

Compared to the third system 100c, the seventh system 100g may omit the fill indicator 128 and may include a turbulence conduit 156 downstream from the eductor 108 comprising turbulence-forming objects on its interior, which may include small perturbations or flow trip posts. The seventh system 100g may include a foaming section 158 comprising a set of one or more foaming screens of at least 200 mesh size perpendicular to the flow direction.

In operation, the mixing region 106 is connected to a motive flow source (i.e., the pressurized first container 102) wherein the pressurized motive gas is first released by a “precision burst” of gas lasting less than 1 second and thereby clearing ambient air from the first precision dose chamber 112, followed by closing the first NC effluent valve 122 and capturing a gas volume sufficient to fill or “charge” the first precision dose chamber 112, which is then “locked off” by closing the first NC influent valve 116, filling the first precision dose chamber 112 with a predetermined volume of specified mass sufficient to produce a single dose from the foaming nozzle 110.

The first and second NC effluent valves 122, 124 may be simultaneously opened permitting gas flow from the first precision dose chamber 112 into the motive opening of the eductor 108. This motive gas flow induces a suction flow in the effluent line of the second container 104, up through the open second NC effluent valve 124 and into the suction opening of the eductor 108 where the first mixture is mixed with the second mixture through the eductor 108. Nitric oxide microbubble foam production may be initiated when motive gas and the suction liquid flows enter and pass through the eductor 108, which may be a Venturi eductor 108 (the Venturi may be part of the eductor 108 or other means precisely engineered for the predetermined dosage by taking into consideration the extrinsic and intrinsic variables such as temperature, ambient pressure, liquid viscosity, flow length (diameters of mixing), venturi cross-sectional areas across the motive flow path, enthalpy change due to throttling, gas volume, density, temperature, and pressure in accordance with the ideal gas law). A further combining and mixing of the flows may occur as the mixed flow passes through the turbulence-forming objects of the turbulence conduit 156, the screens of the foaming section 158 (wherein the turbulent flow experiences intense shear forces as it passes around and through the micro screens of the foaming section 158), and the foaming nozzle 110 and into the ambient. In some implementations, the foaming nozzle 110 may include a flow-straightening portion to straighten the flow of the foam before discharge.

The resultant microbubble foam is comprised of microbubbles containing the motive gases in a predetermined ratio ranging from 0.1 ppm to 20,000 ppm and optimized for the desired treatment protocol such as diabetic ulcers, chronic wounds, and the like. The surfactant and water may be selected in a predetermined ratio to provide a durable foam filled with motive gas that exhibits sufficient viscosity to initially “stick” to horizontal and non-horizontal surfaces, e.g., the skin of a patient.

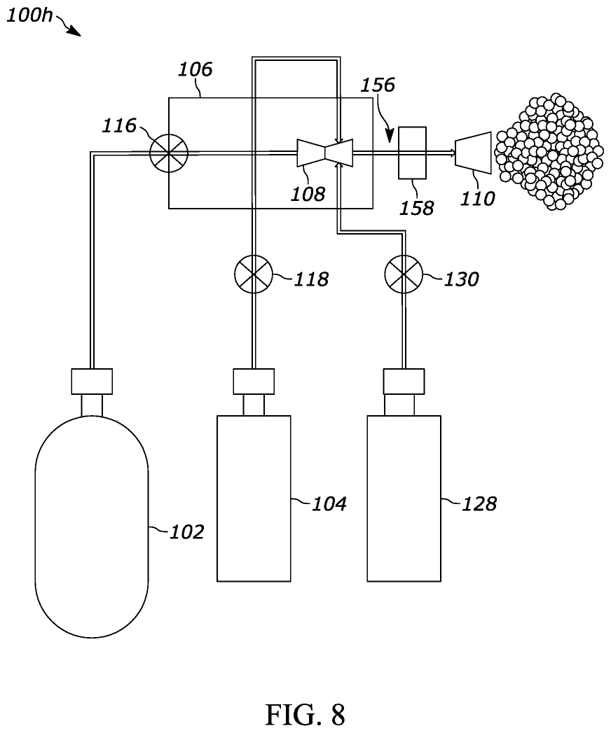

Referring to FIG. 8, an eighth exemplary system for nitric oxide administration 100h is generally shown. The eighth system 100h comprises the first pressurized container 102 containing a mixture of motive gases (i.e., CO2 or/and N2, Ar or other inert gases), which is connected by gas line to the first NC influent valve (or trigger valve) 116 at the mixing region 106.

The second container 104 may contain an acidic liquid, surfactant, and purified water mixture. The third container 128 may contain a surfactant, nitrite, and purified water mixture. Each container 104, 128 may be opened to the atmosphere at the beginning of the dosage preparation process and can be primed before use by purging any ambient or contaminate gases. The eductor 108 may be precisely designed to uptake the desired volumes for a rich foam bolus upon “triggering” of the gas flow. The gas and liquid flows may be turbulently mixed at the confluence of the gas and liquid flow in the eductor 108 before flowing to and through the turbulence conduit 156, the foaming section 158, and the foaming nozzle 110.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed above could be termed a second element, component, region, layer or section without departing from the teachings of the example configurations.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A method for producing a gas-filled medium, comprising:
providing a source of gas;
providing a surfactant solution;
mixing the gas and the surfactant solution in a manner that produces a gas-filled medium; and
applying the gas-filled medium topically to the skin.

2. The method of claim 1, wherein the medium is a cluster of bubbles.

3. The method of claim 2, wherein the bubbles comprise at least one of macro-bubbles, micro-bubbles, nano-bubbles or pico-bubbles.

4. The method of claim 1, wherein the source of the gas is a pressurized vessel.

5. The method of claim 1, wherein the gas comprises one or more of nitric oxide, oxygen, or nitrogen.

6. The method of claim 1, wherein the surfactant is selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.

7. A method of producing a gas-filled medium, comprising:
providing a source of gas;
providing a first solution containing at least Acidic Liquid, Surfactant, and Purified Water;
providing a second solution containing at least a Surfactant, Nitrite, and Purified Water;
using the pressure difference from source of gas to draw out a specific volume of the first and second solutions and enter a holding chamber;
allowing the first and second solutions to mix in the holding chamber and trap the gas;
opening a valve to release the mixed first and second solutions and trapped gas; and
applying the gas-filled medium topically to the skin.

8. The method of claim 7, wherein the medium is a cluster of bubbles.

9. The method of claim 8, wherein the bubbles comprise at least one of macro-bubbles, micro-bubbles, nano-bubbles or pico-bubbles.

10. The method of claim 7, wherein the source of the gas is a pressurized vessel.

11. The method of claim 7, wherein the gas comprises one or more of nitric oxide, oxygen, or nitrogen.

12. The method of claim 7, wherein the surfactant is selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.

13. A system for producing a gas-filled medium, comprising:
a first pressurized vessel containing a gas;
a second vessel containing a surfactant solution;
a dose chamber in fluid communication with the first pressurized vessel;
a mixing chamber in fluid communication with the dose chamber and the second vessel, the mixing chamber being configured to mix the gas flowing out of the dose chamber and the surfactant solution flowing out of the second vessel to produce a gas-filled solution; and
a foaming nozzle comprising mesh screens configured to convert the gas-filled solution into a gas-filled foam medium, the foaming nozzle configured to discharge the gas-filled foam medium.

14. The system of claim 13, wherein the medium is a cluster of bubbles.

15. The system of claim 14, wherein the bubbles comprise at least one of macro-bubbles, micro-bubbles, nano-bubbles or pico-bubbles.

16. The system of claim 13, wherein the gas comprises one or more of nitric oxide, oxygen, or nitrogen.

17. The system of claim 13, wherein the surfactant is selected from the group consisting of cetyl trimethyl ammonium bromide, cetrimonium bromide; dodecylbenzenesulfonic acid, cetylpyridinium chloride, stearalkonium chloride, polyquaternium-7, coco betaine, cocamidopropyl betaine, lauryl dimethyl ammonium chloride, polyquaternium-10, behentrimonium chloride, and cetrimonium chloride or a combination thereof.

18. The system of claim 13, wherein the mixing chamber comprises an eductor configured to receive the gas flowing out of the dose chamber and the surfactant solution flowing out of the second vessel.

19. The system of claim 13, further comprising:
a first valve disposed between the first pressurized vessel and the dose chamber;
a second valve disposed between the dose chamber and the mixing chamber; and
a third valve disposed between the second vessel and the mixing chamber.

20. The system of claim 13, wherein the second vessel is pressurized.
